(19) United States
(12) Patent Application Publication
(10) Pub. No.: US 2004/0248914 A1
(43) Pub. Date: Dec. 9, 2004

(54) Method for the treatment of overactive bladder

(76) Inventors: Russell Bialecki, Delaware, DE (US); Cathy Dantzman, Delaware, DE (US); Keith Herzog, Delaware, DE (US)

Correspondence Address: ASTRA ZENECA PHARMACEUTICALS LP, GLOBAL INTELLECTUAL PROPERTY, 1800 CONCORD PIKE, WILMINGTON, DE, 19850-5437, US

(21) Appl. No.: 10/494,387

(22) PCT Filed: Nov. 1, 2002
(86) PCT No.: PCT/SE02/01990

(30) Foreign Application Priority Data
Nov. 2, 2001 (SE) 0103668-0

(51) Int. Cl. A61K 31/513

(52) U.S. Cl. 514/269

(57) ABSTRACT

Treatment or prevention of OAB or UI in mammals, particularly humans, is disclosed using NK2R binding compounds in accord with structural diagram I:

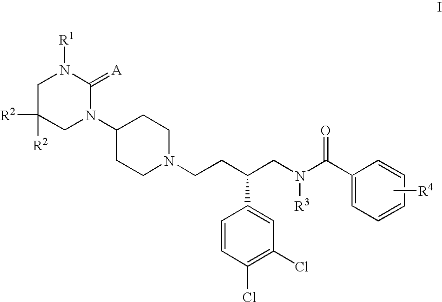

wherein A, R¹, R², R³ and R⁴ are as defined in the specification. Pharmaceutically-advantageous salts of the compounds, methods of use of the compounds, either alone or in combination with other pharmacological agents, and pharmaceutical compositions useful in practicing the methods of the invention are also disclosed.

### FIELD OF THE INVENTION

This invention relates to a method for the treatment and/or prevention of overactive bladder or urinary incontinence and compounds and compositions for the use in the method.

### BACKGROUND

Overactive bladder (“OAB”) is a term for a syndrome that encompasses urge incontinence, urgency and frequency. Urinary incontinence (“UI”) is the involuntary loss of urine that results from an inability of the bladder to retain urine as a consequence of either urge (urge incontinence), or physical or mental stress (stress incontinence).

The normal bladder fills at a physiological rate dictated by the function of the kidneys. The bladder can accommodate large volumes of urine due to the physical properties of the bladder as well as a neural inhibitory system. The inhibitory mechanism is believed to involve inhibition of parasympathetic activity or an increase in sympathetic tone to produce detrusor relaxation and allow filling to occur. During filling the outlet neck of the bladder and urethra are contracted preventing leakage. Voiding or micturition is characterized by a relaxation of the outlet neck and the urethra followed by contraction of the detrusor muscle. When the bladder is empty the detrusor muscle relaxes and the outlet neck and urethra contract to seal off the bladder and maintain continence.

Between 4 and 8% of the total population are estimated to suffer from UI at any point in time, although in most countries, only about 15% of such sufferers are diagnosed. Of those diagnosed only about 70% receive medical treatment. Urge incontinence is more prevalent in the elderly and 80% of the cases are female. Pads and other physical devices are regularly used by a large proportion of incontinent patients not receiving medical treatment. The US market for incontinence pads was estimated at $1.5 billion in 1997.

The muscarinic antagonist oxybutin is prescribed for treatment for OAB in western countries and a second generation muscarinic M3 receptor antagonist, tolterodine, is also marketed for OAB. Propiverine and Flavoxate are prescribed in Japan. Estrogen and progesterone therapy has been studied and is believed to partially alleviate incontinence in some women. Other studies suggest alpha-adrenergic agonists, beta-adrenergic-receptor blocking agents, cholinergic receptor-blocking compounds and cholinergic receptor-stimulating drugs may be beneficial. However, existing therapies are associated with side effects including constipation, visual-accommodation abnormalities, xerothalmia (dry eyes) and a “dry mouth” side effect, which is poorly tolerated by some users and therefore, despite the availability of existing treatments, there is a major unmet and growing need for an effective and acceptable medical treatment for UI and OAB.

### DESCRIPTION OF THE INVENTION

It has now been discovered that certain compounds that bind to the neurokinin 2 receptor (“NK2R”) are useful for the treatment and prevention of overactive bladder (“OAB”) and urinary or urethral incontinence (“UI”). In particular, it has been discovered that compounds that bind to the NK2R receptor useful for the treatment and prevention OAB and UI are certain compounds having a structure in accord with structural diagram I:

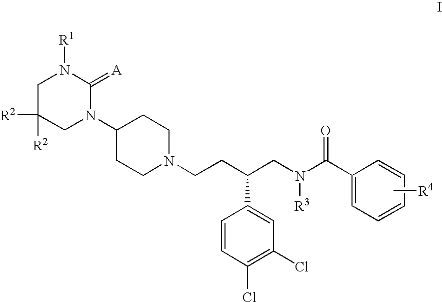

wherein,

A is O or S;

R¹ is selected from H or C₁₋₄alkyl;

R² moieties are independently selected from H or C₁₋₄alkyl;

R³ is selected from C₁₋₄alkyl;

R⁴ is selected from halogen, C₁₋₄alkyl, C₁₋₄alkoxy or cyano,

or a pharmaceutically-acceptable salt thereof with the proviso that R³ is not methyl when R¹,

R² and R⁴ are all H.

More particularly, it has been discovered that compounds in accord with structural diagram I wherein A is O, R¹ and R² are all H, R³ is C₁₋₄alkyl, and R⁴ is selected from H or halo are useful for the treatment and prevention OAB and UI with the proviso that R³ is not methyl when R⁴ is H.

Still more particularly, it has been discovered that compounds in accord with structural diagram I wherein A is O, R¹, R² and R⁴ are all H, and R³ is C₂₋₄alkyl are useful for the treatment and prevention OAB and UI.

Most particular compounds useful for the treatment and prevention OAB and UI are those exemplified herein.

Compounds of the invention possesses NK2R binding properties and certain such compounds selectively inhibit the contraction of bladder tissues. Surprisingly, it has been found that certain closely related compounds activate the contraction of bladder tissues induced by BANK. One such compound is one wherein, by reference to structural diagram I, A is S, R¹, R² and R⁴ are H and R³ is methyl.

In one aspect, the Invention provides a method comprising treating or preventing OAB or UI in a subject, particularly in a human, with a compound in accord with structural diagram I and, more particularly, a method that comprises treating with a therapeutically-effective amount of a compound having a structure in accord with structural diagram I.

In a second aspect, the Invention provides a compound of the present invention, for the treatment and prevention of OAB or UI in mammals, and in humans in particular.

In a third aspect, the Invention provides pharmaceutically-acceptable salts of a compound of the present invention and compositions containing said compound or pharmaceutically-acceptable salts thereof.

In a particular aspect, the Invention provides a method comprising treating or preventing OAB or UI in a subject, particularly in a human, with a therapeutically-effective amount of a compound having a structure in accord with structural diagram I that inhibits bladder contractions.

In another aspect, the Invention provides a method for the treatment and prevention of OAB or UI in mammals and humans in particular comprising treating a subject in need thereof with a therapeutically-effective amount of an NK2R binding-compound in combination with another therapeutic agent.

In yet another aspect, the Invention provides a method for the treatment and prevention of OAB or UI in mammals and humans in particular comprising treating a subject in need thereof with a therapeutically-effective amount of an NK2R antagonist in combination with an estrogenic agent and/or a progestational substance, and with or without supplementation with an alpha-adrenergic agonist, beta-adrenergic receptor blocking agent, cholinergic-receptor blocking compound or a cholinergic-receptor-stimulating drug.

In a further aspect, the Invention provides a pharmaceutical composition useful in the practice of the methods of the Invention comprising a compound in accord with structural diagram I and a pharmaceutically-acceptable excipient or diluent.

In all aspects of the invention pharmaceutically-acceptable salts contemplated to be within the scope of the invention are salts such as a hydrochloride, sulphate, tosylate, mesylate, napsylate, besylate, phosphate, salicylate, tartrate, lactate, citrate, benzoate, succinate, fumerate, acetate or a maleate.

It is an object of the Invention to provide a method for the treatment of OAB or UI comprising use of a compound, having a structure in accord with structural diagram I as described heretofore.

It is another object of the Invention to provide a method comprising use of a compound of the present invention for the prevention of OAB or UI.

While the methods of the Invention are applicable to mammals in general they are applicable to humans in particular.

Therefore, it is an object of the Invention to provide a method comprising treating a human patient suffering from OAB or UI and in need of treatment therefore with a therapeutically-effective amount of a compound of the present invention.

Another object of the Invention is to provide a compound in accord with structural diagram I useful for the treatment or prevention of OAB or UI.

A further object of the Invention is to provide pharmaceutically-acceptable salts, compositions, mixtures and the like of said compound useful for the treatment or prevention of OAB or UI.

A particular object of the invention is to provide a method of treating a human patient having OAB or UI, comprising administering an effective OAB or UI treatment amount of a compound having a structure in accord with structural diagram I to the patient.

Another particular object of the invention is to provide a method wherein a compound having a structure in accord with structural diagram I is in the form of a pharmaceutically-acceptable salt thereof.

In methods of the invention treatment is contemplated to be administered in any physiologically-acceptable manner, such as by topical application, ingestion, inhalation, insufflation or injection.

In methods of the invention a compound of the present invention is contemplated to be in a form such as a capsule, a tablet, an aqueous solution, an aqueous suspension, a non-aqueous suspension, a suppository, an aerosol or a powder.

Treatment of overactive bladder (“OAB”) a term generally used, and used herein, for a syndrome that encompasses urinary urge incontinence, urgency and frequency or urinary incontinence (“UI”), the involuntary loss of urine that results from an inability of the bladder to retain urine as a consequence of either urge (urge incontinence), or physical or mental stress (stress incontinence), is an object of the Invention.

Therefore, it is an object of the Invention to provide a method for treating a human patient suffering from OAB or UI.

A particular object of the method of the invention for treating OAB or UI, as contemplated herein, is administration of a therapeutically-effective amount of a compound in accord with structural diagram I.

Another object of the Invention is to provide a compound in accord with structural diagram I useful for the treatment or prevention of OAB or UI.

A further object of the Invention is to provide pharmaceutically-acceptable salts, compositions, mixtures and the like of a compound of the Invention useful for the treatment or prevention of OAB or UI.

A particular object of the invention is to provide a method of treating a human patient having OAB or UI comprising administering an effective OAB or UI treatment amount of (S)-N-[2-(3,4-dichlorophenyl)-4-[4-(2-oxoperhydropyrimidin-1-yl)piperidino]butyl]-N-ethylbenzamide to the patient.

Another particular object of the invention is to provide a method wherein (S)-N-[2-(3,4-dichlorophenyl)-4-[4-(2-oxoperhydropyrimidin-1-yl)piperidino]butyl]-N-ethylbenzamide is in the form of a pharmaceutically-acceptable salt thereof.

In methods of the invention treatment is contemplated to be administered by any physiologically-acceptable route for example, by dermal, sublingual, or rectal topical application; by intraperitoneal, parenteral, intradermal or subcutaneous injection; by ingestion of a capsule, a tablet or a liquid solution or suspension; or by inhalation or insufflation of a powder of aerosol.

Generally, it is contemplated that pharmaceutical compositions of the invention will be formulated so as to permit administration by a physiologically-acceptable route. In methods of the invention compounds are contemplated to be administered in the form such as a capsule, a tablet, an aqueous solution, an aqueous suspension, a non-aqueous suspension, a suppository, an aerosol or a powder.

In certain methods of the invention it is contemplated that compounds will be administered in combination with one or more other therapeutic agents. Such agents are contemplated to be estrogenic agents, progestational substances, alpha-adrenergic agonists, beta-adrenergic-receptor-blocking agents, cholinergic-receptor-blocking agents or cholinergic-receptor-stimulating agents. However, it will be apparent to those of skill in the art that the compounds of the invention can be co-administered with any therapeutic or prophylactic agent and/or medicament or combination thereof that is medically-compatible therewith.

The invention is contemplated to encompass pharmaceutical compositions comprising compounds of the invention together with al least one pharmaceutically-acceptable excipient or diluent.

The invention is also envisioned to encompass pharmaceutical compositions that include agents such as estrogenic agents, progestational substances, alpha-adrenergic agonists, beta-adrenergic-receptor-blocking agents, cholinergic-receptor-blocking agents or cholinergic-receptor-stimulating agents.

Pharmaceutical compositions contemplated to fall within the scope of the invention include those having forms such as capsules, tablets, aqueous solutions, aqueous suspensions, non-aqueous suspensions, suppositories, aerosols and powders.

Further aspects, objects and advantages of this Invention will become apparent to those skilled in the art upon study of the specification and the appended claims.

However, it will be appreciated that when used in the treatment of OAB, UI or related disease, a compound of the Invention is contemplated to be administered as an appropriate pharmaceutical composition which comprises a compound of the Invention or a pharmaceutically-acceptable salt of such a compound, such as a chloride, sulphate, tosylate, mesylate, napsylate, besylate, phosphate, salicylate, tartrate, lactate, citrate, benzoate, succinate, acetate, maleate, or the like, together with a pharmaceutically-acceptable diluent or carrier. Such salts are prepared by methods known to those of skill in the art. The form of a pharmaceutical composition is adapted for the particular route of administration chosen. Such forms include, for example, tablets, capsules, solutions or suspensions for oral administration; solutions or suspensions for topical administration; suppositories for rectal administration; sterile solutions or suspensions for administration by intravenous or intramuscular infusion or injection; aerosols or nebulizer solutions or suspensions for administration by inhalation; or powders together with pharmaceutically-acceptable solid diluents such as lactose for administration by insufflation.

For oral administration a tablet or capsule containing therapeutically-effective amount from 0.1 mg up to 250 mg (and typically 5 to 100 mg) of a compound of the Invention may conveniently be used. For administration by inhalation, a compound of the Invention will be administered to humans in a daily dose range of, for example, 5 to 100 mg, in a single dose or divided into two to four daily doses. Similarly, for intravenous or intramuscular injection or infusion a sterile solution or suspension containing up to 10% w/w (and typically 0.05 to 5% w/w) of a compound of the Invention may conveniently be used.

The dose of a compound of the Invention to be administered will necessarily be varied according to principles well known in the art, taking account of the route of administration and the severity of the condition and the size and age of the patient under treatment. General, it is contemplated that a compound of the Invention will be administered as a dose within the range of about 0.01 to about 25 mg/kg, and more particularly as a dose within the range 0.1 to 5 mg/kg. It will be understood that generally equivalent amounts of an N-oxide or a pharmaceutically-acceptable salt or a quaternary ammonium salt of a compound of the Invention may be used.

### EXAMPLES

As used herein, unless stated otherwise:

(i) temperatures are given in degrees Celsius (“° C.”); operations were carried out at room or ambient temperature, that is, at a temperature in the range of 18-25° C.;

(ii) organic solutions were dried over anhydrous MgSO₄; evaporation of solvent was carried out using a rotary evaporator under reduced pressure (600-4000 pascals; 4.5-30 mm Hg) with a bath temperature of up to 60° C.;

(iii) chromatography, means flash chromatography; reversed phase chromatography, means flash chromatography over octadecylsilane (“ODS”) coated support having a particle diameter of 32-74μ, known as “PREP-40-ODS” (Art 731740-100 from Bodman Chemicals, Aston, Pa., USA); Thin layer chromatography (“TLC”) was carried out on silica gel plates;

(iv) in general, the course of reactions was followed by TLC and reaction times are given for illustration only;

(v) melting points are uncorrected and “dec” indicates decomposition; the melting points given are those obtained for the materials prepared as described; polymorphism may result in isolation of materials with different melting points in some preparations;

(vi) final products had satisfactory proton nuclear magnetic resonance (“NMR”) spectra;

(vii) yields are given for illustration only and are not necessarily those which may be obtained by diligent process development; preparations were repeated if more material was required;

(viii) when given, NMR data is in the form of delta values for major diagnostic protons, given in parts per million (“ppm”) relative to tetramethylsilane (“TMS”) as an internal standard, determined at 300 MHz using perdeuterio dimethyl sulfoxide (“DMSO-d6”) as solvent; conventional abbreviations for signal shape are used; coupling constants (J) are given in Hz; Ar designates an aromatic proton when such an assignment is made;

(ix) chemical symbols have their usual meanings; SI units and symbols are used;

(x) reduced pressures are given as absolute pressures in pascals (“Pa”); elevated pressures are given as gauge pressures in bars;

(xi) solvent ratios are given in volume:volume (“v/v”) terms;

(xii) mass spectra (“MS”) were run with an electron energy of 70 electron volts in the electron impact (“EI”) mode using a direct exposure probe; where indicated ionization was effected by chemical ionization (“CI”) or fast atom bombardment (“FAB”); values for m/z are given; generally, only ions which indicate the parent mass are reported; and

(xiii) LC/MS was detected by a diode ray detector. The analysis was conducted with a Zorbax 50 mm×2.1 mm stable bond C8 analytical column. Solvent A was 0.05% trifluoroacetic acid in water. Solvent B was 90% acetonitrile 9.95% water and 0.05% trifluoroacetic acid. The flow rate was 1.4 mL/minute ramping from 5% B to 90% B in 3 minutes. Retention times are given in minutes. The ionization method was APCI, or atmospheric pressure chemical ionization. Generally, only ions which indicate the parent ions are reported.

### Chemical Examples

### Example 1

(S)-N-[2-(3,4-Dichlorophenyl)-4-[4-(2-oxo-5,5-dimethyl-perhydropyrimidin-1-yl)piperidino]butyl]-N-methylbenzamide.

(S)-N-[2-(3,4-dichlorophenyl)-4-oxopropyl]-N-methylbenzamide (0.622 g) in methanol (8.0 mL) was added to a solution of 4-(2-oxo-5,5-dimethylperhydropyrimidin-1-yl)piperidine (0.400 g) and acetic acid (0.11 mL) in methanol (8.0 mL). After 5 minutes, sodium cyanoborohydride (0.119 g) in methanol (8.0 mL) was added in a single portion. After being stirred overnight, the reaction mixture was diluted with aqueous sodium bicarbonate, stirred for 30 minutes, and extracted with dichloromethane. The separated organic layer was dried, evaporated, and purified by chromatography, with dichloromethane: methanol (95:5) as eluent. The resulting oil, which began to crystallize upon standing, was suspended in ether and filtered to give the title compound as a white solid (0.720 g). MS: m/z=545(M+1); Analysis for C₂₉H₃₈Cl₂N₄O₂: Calculated: C, 63.84; H, 7.02; N, 10.26; Found: C, 63.95; H, 6.95; N, 10.15.

The intermediate 4-(2-oxo-5,5-dimethylperhydropyrimidin-1-yl)-piperidine was synthesized as follows:

1a. 1-Benzyloxycarbonyl-4-(3-amino-2,2-dimethylpropylamino)-piperidine.

1-Benzyloxycarbonyl-4-oxo-piperidine (12.0 g) in methanol (72 mL) was added to a stirred solution of 2,2-dimethyl-1,3-propanediamine (5.2 mL) and acetic acid (8.8 mL) in methanol (72 mL). After 15 minutes, sodium cyanoborohydride (9.7 g) in methanol (72 mL) was added in a single portion. After being stirred overnight, the reaction mixture was evaporated; and the residue was dissolved in 1 N hydrochloric acid (100 mL). Concentrated hydrochloric acid was added dropwise and stirring was continued until the evolution of gas ceased. The acidic aqueous mixture was washed with dichloromethane, basified to pH 10 with 10 N sodium hydroxide, and extracted with dichloromethane. The dichloromethane extracts were dried and evaporated to give the title compound as a viscous oil. NMR (CD₃OD): 7.34 (m,5), 5.10 (s,2), 4.08 (m,2), 2.93 (m,2), 2.57 (m,1), 2.46 (s,2), 2.44 (s,2), 1.89 (m,2), 1.27 (m,2), 0.89 (s,6).

1b. 1-Benzyloxycarbonyl-4-(2-oxo-5,5-dimethylperhydropyrimidin-1-yl)-piperidine.

A solution of -benzyloxycarbonyl-4-(3-amino-2,2-dimethylpropylamino)-piperidine (3.02 g) and 1,1′-carbonyldiimidazole (2.19 g) in chloroform (40 mL) was refluxed for 3 hours. The reaction mixture was diluted with dichloromethane and washed sequentially with 1 N hydrochloric acid and aqueous sodium bicarbonate. The separated organic phase was dried, evaporated, triturated from ether, and filtered to give the urea as a white solid (1.72 g). MS: m/z=346(M+1); NMR (CD₃OD): 7.34 (m,5), 5.10 (s,2), 4.35 (m,1), 4.23 (m,2), 2.87 (m,6), 1.58 (m,4), 1.00 (s,6).

1c. 4-(2-Oxo-5,5-dimethylperhydropyrimidin-1-yl)piperidine.

A solution of 1-benzyloxycarbonyl-4-(2-oxo-5,5-dimethylperhydropyrimidin-1-yl)piperidine(1.85 g) and 20% palladium hydroxide on carbon (0.340 g) in ethanol (30 mL) was stirred overnight under 1 bar of hydrogen. The reaction mixture was filtered through diatomaceous earth and the filtrate was evaporated to give the title compound (0.950 g) as a white solid. MS: m/z=212(M+1); NMR (CD₃OD): 4.28 (m,1), 3.10 (m,2), 2.92 (m,2), 2.89 (m,2), 2.66 (m,2), 1.59 (m,4), 1.03 (s,6).

### Example 2

(S)-N-[2-(3,4-Dichlorophenyl)-4-[4-(3-ethyl-2-oxoperhydro-pyrimidin-1-yl)-piperidino]butyl]-N-methylbenzamide citrate.

(S)-N-[2-(3,4-Dichlorophenyl)-4-oxobutyl]-N-methylbcnzamide (0.883 g) in methanol (10.0 mL) was added to a solution of 4-(3-ethyl-2-oxoperhydro-pyrimidin-1-yl)-piperidine (0.498 g) and acetic acid (0.145 mL) in methanol (10.0 mL). After 5 minutes, sodium cyanoborohydride (0.159 g) in methanol (10.0 mL) was added in a single portion. After being stirred for 3.5 hours, the reaction mixture was diluted with aqueous sodium bicarbonate, stirred for 30 minutes, and extracted with dichloromethane. The separated organic layer was dried, evaporated, and purified by chromatography, with dichloromethane:methanol (95:5) as eluent. The resulting oil (0.970 g) and citric acid (0.352 g) were dissolved in methanol and evaporated to give the title compound as a white solid. MS: m/z=545(M+1); Analysis for C₂₉H₃₈C₁₂N₄O₂.1.00 C₆H₈O₇: Calculated: C, 56.98; H, 6.28; N, 7.59; Found: C, 56.66; H, 6.31; N, 7.57.

The intermediate 4-(3-ethyl-2-oxoperhydropyrimidin-1-yl)-piperidine was prepared as follows:

2a. 1-Benzyloxycarbonyl-4-(3-ethyl-2-oxoperhydropyrimidin-1-yl)-piperidine.

Potassium tert-butoxide (19.3 mL, 1 M in tetrahydrofuran) was added to a solution of 1-benzyloxycarbonyl-4-(2-oxoperhydro-pyrimidin-1-yl)piperidine (3.06 g) in tetrahydrofuran (88 mL). Iodoethane (2.4 mL) was then added, and the reaction mixture was stirred for 30 minutes. The reaction mixture was diluted with dichloromethane, washed with water, and purified by chromatography, with dichloromethane:methanol (gradient 98:2, 90:10) as eluent. The product was triturated from ether and filtered to give the N-methyl compound as a white solid. MS: m/z=346(M+1); NMR (CDCl₃): 7.34 (m,5), 5.12 (s,2), 4.54 (m,1), 4.26 (m,2), 3.38 (q,2, J=7.1), 3.22 (m,2), 3.11 (m,2), 2.86 (m,2), 1.90 (m,2), 1.60 (m,4), 1.10 (t,3, J=7.1).

2b. 4-(3-Ethyl-2-oxoperhydropyrimidin-1-yl)piperidine.

A solution of 1-benzyloxycarbonyl-4-(3-ethyl-2-oxoperhydropyrimidin-1-yl)piperidine (1.85 g) and 20% palladium hydroxide on carbon (0.340 g) in ethanol (30 mL) was stirred overnight under 1 bar of hydrogen. The reaction mixture was filtered through diatomaceous earth and the filtrate was evaporated to give the title compound (0.950 g) as a 10 viscous oil. MS: m/z=212(M+1); NMR (CDCl₃): 4.45 (m,1), 3.38 (q,2, J=7.1), 3.17 (m,6), 2.72 (m,2), 2.15 (m,1), 1.91 (m,2), 1.62 (m,4), 1.10 (t,2, J=7.1).

### Example 3

(S)-N-{2-(3,4-Dichloro-phenyl)-4-[4-(2-oxo-tetrahydro-pyrimidin-1-yl)-piperidin-1-yl]-butyl}-N-ethyl-benzamide free base.

(5)-N-[2-(3,4-Dichloro-phenyl)-4-oxo-butyl]-N-ethyl-benzamide (0.883 g) in methanol (10.0 mL) was added to a solution of 1-piperidin-4-yl-tetrahydro-pyrimidin-2-one (0.498 g) and acetic acid (0.145 mL) in methanol (10.0 mL). After 5 minutes, sodium cyanoborohydride (0.159 g) in methanol (10.0 mL) was added in a single portion. The reaction mixture was stirred for 3.5 hours, diluted with aqueous sodium bicarbonate, stirred for 30 minutes, and extracted with dichloromethane. The separated organic layer was dried, evaporated, and the title compound purified by chromatography, with dichloromethane:methanol (95:5) as eluent.

The intermediate, (S)-N-[2-(3,4-dichloro-phenyl)-4-oxo-butyl]-N-ethyl-benzamide, was prepared as follows.

3a. (5)-Benzoic acid 4-benzoylamino-3-(3,4-dichloro-phenyl)-butyl ester.

Benzoyl chloride (168.3 g) in dichloromethane (200 mL) was added dropwise to a solution of (S)-4-amino-3-(3,4-dichloro-phenyl)-butan-1-ol (140.0 g) and triethylamine (121.4 g) in dichloromethane (1400 mL) at 0° C. The solution was stirred at ambient temperature overnight. The resultant white precipitate was filtered the next morning, washed with dichloromethane and resultant white solid was discarded. The mother liquor was washed with saturated aqueous sodium bicarbonate and the separated organic phase was dried and evaporated. The amber oil was purified by flash chromatography with dichloromethane:methanol (gradient 100, 90:10) as eluent. The title compound was isolated in two fractions. One fraction (131.2 g) LC/MS exhibited one peak at 2.98 rt, m/z=442(M+1); NMR (CD₃OD): 8.44 (m,1), 7.82 (d,1,J=8.1), 7.67 (d,1,J=7.5), 7.46 (m, 10), 7.21 (dd,1,J=1.7,8.3), 4.27 (m,2), 3.57 (m,2), 3.24 (m,1), 2.16 (m,2). The other fraction (127.7 g) was further purified by chromatography.

3b. (S)-N-[2-(3,4-Dichloro-phenyl)-4-hydroxy-butyl]-benzamide.

A solution of (S)-benzoic acid 4-benzoylamino-3-(3,4-dichloro-phenyl)-butyl ester (127.7 g) in tetrahydrofuran (800 mL) and aqueous sodium hydroxide (800 mL of 2.5 normal sodium hydroxide) was heated at reflux overnight. The next day the reaction was concentrated in vacuo, dissolved in dichloromethane, and washed with water and brine. The separated organic layer was dried, evaporated, and purified by chromatography, with dichloromethan:methanol (gradient 98:2, 95:5) as eluent, to yield the title compound as a yellow oil (85.4 g). LC/MS: one peak 2.18 rt, m/z=338(M+1); NMR (CD₃OD): 7.66 (m,2), 7.43 (m,5), 7.20 (m,1), 3.59 (m,2), 3.30 (m,2), 3.18 (m,1), 2.00 (m,1), 1.86 (m,1).

3c. (S)-N-[4-(tert-Butyl-dimethyl-sianyloxy)-2-(3,4-dichloro-phenyl)-butyl]-benzamide.

4-Dimethylaminopyridine (13.0 g) and triethylamine (30.15 g) were dissolved in a solution of (S)-N-[2-(3,4-Dichloro-phenyl)-4-hydroxy-butyl]-benzamide (71.6 g) and dichloromethane (900 mL). To this mixture was added portion-wise, tert-butyldimethylchlorosilane. The reaction was then diluted with dichloromethane (200 mL). After stirring for 3 hours, the mixture was further diluted with dichloromethane, and washed with dilute aqueous hydrochloric acid, water and aqueous sodium bicarbonate. The separated organic layer was dried and evaporated to an amber oil (105.3 g). LC/MS: one peak 3.43 rt, m/z=452(M+1).

3d. (S)-N-[4-(tert-Butyl-dimethyl-silanyloxy)-2-(3,4-dichloro-phenyl)-butyl]-N-ethyl-benzamide

A slurry of sodium hydride (11.16 g) was prepared in dimethylformamide (1000 mL) and the slurry cooled in an ice bath. To the stirring slurry was added the (S)-N-[4-(tert-butyl-dimethyl-silanyloxy)-2-(3,4-dichloro-phenyl)-butyl]-benzamide as a solution in dimethylformamide (500 mL). The ice bath was removed and the solution was stirred and permitted to warm to ambient temperature for 1 hour. The reaction mixture was cooled in an ice bath before neat ethyl iodide (43.59 g) was added. The reaction mixture was stirred for 30 minutes in the ice bath, the ice bath was removed and the solution stirred for another 2 hours and permitted to warm to ambient temperature. A solution of water (200 mL) and dimethylformamide (200 mL) was added and the entire reaction mixture was concentrated in vacuo. The concentrated material was diluted with water and washed successively with ethyl acetate. The ethyl acetate layers were combined and washed with water and aqueous sodium bicarbonate, dried and evaporated to an amber oil (120.5 g). This material was not analyzed further and taken to the next step. LC/MS: two peaks 2.41 rt 20%, m/z=366(M+1 of byproduct removed later in synthesis by chromatography), and 3.61 rt 80%, m/z=480(M+1).

3e. (S)-N-[2-(3,4-Dichloro-phenyl)-4-hydroxy-butyl]-N-ethyl-benzamide.

To a solution of (S)-N-[4-(tert-butyl-dimethyl-silanyloxy)-2-(3,4-dichloro-phenyl)-butyl]-N-ethyl-benzamide (120.5 g, 212 millimoles in theory from the (S)-N-[2-(3,4-dichloro-phenyl)-4-hydroxy-butyl]-benzamide) in tetrahydrofuran (1000 mL) was added a solution of tetrabutylammonium fluoride (1.0 molar in tetrahydrofuran, 254 mL). After stirring overnight the solution was concentrated in vacuo, diluted with dichloromethane and washed with aqueous sodium bicarbonate. The separated organic layer was dried and purified by chromatography, with dichloromethane:methanol (gradient 98:2, 90:10) as eluent, to give the alcohol as an oil (96% yield over three steps). LC/MS: one peak 2.33 rt, m/z=366(M+1).

3f. (S)-N-[2-(3,4-Dichloro-phenyl)-4-oxo-butyl]-N-ethyl-benzamide.

To a solution of dimethylsulfoxide (82.3 mL) in dichloromethane (700 mL) at −78° C., was added oxalyl chloride (50.6 mL) in dichloromethane (400 mL). After the addition was complete, the solution was stirred for another 30 minutes at −78° C. A solution of (S)-N-[2-(3,4-dichloro-phenyl)-4-hydroxy-butyl]-N-ethyl-benzamide (106.4 g) in dichloromethane (400 mL) and dimethylsulfoxide (10 mL) was then added dropwise maintaining the internal temperature below −60° C. The solution was stirred at −78° C. for one hour. The temperature was allowed to rise to −50° C. and that temperature was maintained for 30 minutes of stirring. The reaction mixture was cooled to −78° C. and stirred for another hour. Triethylamine (202 mL) was added dropwise to this solution, after which the ice bath was removed and the solution was stirred overnight warm permitted to warm to ambient temperature. The mixture was diluted with dichloromethane, washed successively with dilute aqueous hydrochloric acid, water, and aqueous sodium bicarbonate. The separated organic layer was dried, evaporated, and purified by chromatography, with dichloromethane:ethylacetate (85:15) as eluent, to give the title compound as an oil (101.9 g). LC/MS: broad peak 2.42 rt 364(M+1), one small peak <5% 394(M+).

### Example 4

(S)-N-[2-(3,4-Dichlorophenyl)-4-[4-(2-oxoperhydro-pyrimidin-1-yl)piperidino]butyl]-4-fluoro-N-methylbenzamide citrate.

4-Fluorobenzoyl chloride (0.115 mL) was added to a solution of (S)-N-[2-(3,4-dichlorophenyl)-4-[4-(2-oxoperhydropyrimidin-1-yl)piperidino]-butyl]-N-methylamine (0.400 g) and pyridine (0.16 mL) in dichloromethane (10 mL) at −30° C. The reaction mixture was warmed to ambient temperature and stirred for 1 hour. The mixture was diluted with dichloromethane, washed (aqueous sodium bicarbonate, saturated aqueous copper(II) sulfate), dried, and evaporated. The product was purified by chromatography, with dichloromethane:methanol (gradient 98:2, 80:10) as eluent. The purified product (0.350 g) and citric acid (0.126 g) were dissolved in methanol and evaporated to give the title compound as a glass which was scraped out as a white solid (0.450 g). MS: m/z=535(M+1); Analysis for C₂₇H₃₃Cl₂FN₄O₂.1.10 C₆H₈)₇.0.10 (C₂H₅)₂O.0.70 H₂O: Calculated: C, 53.25; H, 5.80; N, 7.30; Found: C, 53.22; H, 5.70; N, 7.30.

The intermediate (5)-N-[2-(3,4-dichlorophenyl)-4-[4-(2-oxoperhydro-pyrimidin-1-yl)piperidino]butyl]-N-methylamine was prepared as follows:

4a. tert-Butyl (S)-N-[2-(3,4-dichlorophenyl)-4-hydroxybutyl]-N-methylcarbamate.

Di-tert-butyl dicarbonate (21.6 g) in dichloromethane (125 mL) was added dropwise to a solution of (S)-N-methyl-2-(3,4-dichlorophenyl)-4-hydroxybutylamine (25.0 g) in dichloromethane (125 mL) over a period of 30 minutes. After being stirred for 3 hours, the reaction mixture was washed (0.1 N hydrochloric acid, aqueous sodium bicarbonate), dried, and evaporated. The product was purified by chromatography, with dichloromethane:ether (2:1) as eluent, to give the tert-butyl ester as an oil (33.0 g) that crystallized upon standing.

4b. tert-Butyl (S)-N-[2-(3,4-dichlorophenyl)-4-oxobutyl)-N-methylcarbamate.

To a solution of oxalyl chloride (1.3 mL) in dichloromethane (30 mL) at −78° C. was added dimethylsulfoxide (2.1 mL) in dichloromethane (10 mL), followed tert-butyl LS)-N-[2-(3,4-dichlorophenyl)-4-hydroxybutyl]-N-methyl-carbamate (3.2 g) in dichloromethane (15 mL) within 5 minutes. After 15 minutes, triethylamine (8.2 mL) was added, and the reaction mixture was warmed to ambient temperature. The mixture was diluted with dichloromethane, and washed with dilute aqueous hydrochloric acid, water, and aqueous sodium bicarbonate. The separated organic layer was dried, evaporated, and used in the next reaction (below) without further purification.

4c. tert-Butyl (5)-N-[2-(3,4-dichlorophenyl)-4-[4-(2-oxoperhydro-pyrimidin-1-yl)piperidino]butyl]-N-methylcarbamate.

tert-Butyl (S)-N-[2-(3,4-dichlorophenyl)-4-oxobutyl]-N-methylcarbamate (0.883 g) in methanol (10.0 mL) was added to a solution of 4-(2-oxoperhydropyrimidin-1-yl)-piperidine (0.498 g) and acetic acid (0.145 mL) in methanol (10.0 mL). After 5 minutes, sodium cyanoborohydride (0.159 g) in methanol (10.0 mL) was added in a single portion. After being stirred for 3.5 hours, the reaction mixture was diluted with aqueous sodium bicarbonate, stirred for 30 minutes, and extracted with dichloromethane. The separated organic layer was dried, evaporated, and chromatographed, with dichloromethane:methanol (95:5) as eluent. The resulting oil (0.970 g) and citric acid (0.352 g) were dissolved in methanol and evaporated to give the title compound as a gum.

4d. (S)-N-[2-(3,4-Dichlorophenyl)-4-[4-(2-oxoperhydropyrimidin-1-yl)piperidino]butyl]-N-methylamine.

Trifluoroacetic acid (7.5 mL) was added to a solution of tert-butyl (5)-N-[2-(3,4-dichlorophenyl)-4-[4-(2-oxoperhydropyrimidin-1-yl)piperidino]-butyl]-N-methylcarbamate (5.1 g) in dichloromethane (200 mL). After 30 minutes, additional trifluoroacetic acid (7.5 mL) was added, and the reaction mixture was stirred for 4 hours. The mixture was washed with 1 N sodium hydroxide (250 mL), dried, and evaporated to give the title compound as a gum (3.8 g). MS: m/z=413(M+1).

### Example 5

(S)-N-[2-(3,4-Dichlorophenyl)-4-[4-(2-thioxoperhydro-pyrimidin-1-yl)-piperidino]butyl]-N-methylbenzamide dihydrochloride.

A stirred solution of (S)-N-[4-[4-(3-aminopropylamino)-piperidino]-2-(3,4-dichloro-phenyl)butyl]-N-methylbenzamide (0.356 g) and 1,1′-thiocarbonyldiimidazole in chloroform (6 mL) was stirred overnight at room temperature. The reaction mixture was diluted with dichloromethane, washed (aqueous sodium bicarbonate), dried, evaporated, and purified by chromatography, with dichloromethane:methanol (gradient 98:2, 90:10) as eluent. The resulting material was dissolved in dichloromethane, precipitated as the hydrochloride salt with ethereal hydrogen chloride, evaporated, and placed under high vacuum overnight to give the title compound as a white solid. MS: m/z=533(M+1); Analysis for C₂₇H₃₄Cl₂N₄OS.2.30 HCl.0.10 (C₂H₅)₂O: Calculated: C, 52.67; H, 6.01; N, 8.96; Found: C, 52.57; H, 6.11; N, 8.84.

The intermediate, (S)-N-[4-[4-(3-aminopropylamino)piperidino]-2-(3,4-dichlorophenyl)butyl]-N-methylbenzamide was prepared as follows:

5a. 1-Benzyloxycarbonyl-4-(3-aminopropylamino)piperidine.

1-Benzyloxycarbonyl-4-oxo-piperidine (12.0 g) in methanol (72 mL) was added to a stirred solution of 1,3-diaminopropane (5.2 mL) and acetic acid (8.8 mL) in methanol (72 mL). After 15 minutes, sodium cyanoborohydride (9.7 g) in methanol (72 mL) was added in a single portion. After being stirred overnight, the reaction mixture was evaporated; and the residue was dissolved in 1 N hydrochloric acid (100 mL). Concentrated hydrochloric acid was added dropwise and stirring was continued until the evolution of gas ceased. The acidic aqueous mixture was washed with dichloromethane, basified to pH 10 with 10 N sodium hydroxide, and extracted with dichloromethane. The dichloromethane extracts were dried and evaporated to give the title compound as a viscous oil. MS: m/z=292(M+1); NMR (CD₃OD): 7.34 (m,5), 5.10 (s,2), 4.13 (m,2), 2.86 (m,2), 2.65 (m,5), 1.90 (m,2), 1.65 (m,2), 1.23 (m,2).

5b. 1-Benzyloxycarbonyl-4-[2,2,2-trifluoroacetyl)-[3-(2,2,2-trifluoroacetylamino)propyl]amino]piperidine.

Trifluoroacetic anhydride (10.5 mL) was added to a solution 1-benzyloxy-carbonyl-4-(3-aminopropylamino)piperidine(7.5 g) and triethylamine (8.3 mL) in chloroform (90 mL) at 0° C. After being stirred overnight, the reaction mixture was diluted with dichloromethane, washed (1 N hydrochloric acid, aqueous sodium bicarbonate), dried, evaporated, and purified by chromatography, with dichloromethane:methanol (98:2) as eluent, to give the trifluoroacetylated piperidine as a viscous oil. NMR: 7.36 (m,5), 5.14 (s,2), 4.35 (m,2), 3.93 (m,1), 3.35 (m,4), 2.83 (m,2), 1.87-1.74 (m,6); MS: m/z=484(M+1).

5c. 4-[(2,2,2-Trifluoroacetyl)[3-(2,2,2-trifluoroacetylamino)propyl]amino]piperidine.

A solution of 1-benzyloxycarbonyl-4-[(2,2,2-trifluoroacetyl)-[3-(2,2,2-trifluoroacetyl-amino)propyl]amino]piperidine(1.85 g) and 20% palladium hydroxide on carbon (0.340 g) in ethanol (30 mL) was stirred overnight under 1 bar of hydrogen. The reaction mixture was filtered through diatomaceous earth and the filtrate was evaporated to give the title compound (0.950 g) as a viscous oil. NMR (CD₃OD): 4.39 (m,1), 3.98 (m,1), 3.30 (m,3), 2.95 (m,1), 2.82 (m,1), 2.65 (m,2), 2.01 (m,2), 1.75 (m,2), 1.32 (m,2); MS: m/z=350(M+1).

5d. (S)-N-[2-(3,4-Dichlorophenyl)-4-[4-[(2,2,2-trifluoroacetyl)-[2-(2,2,2-trifluoroacetyl-amino)ethyl]amino]piperidino]butyl]-N-methylbenzamide.

(S)-N-[2-(3,4-Dichlorophenyl)-4-oxobutyl]-N-methylbenzamide (0.823 g) in methanol (4 mL) was added to a solution of 4-[(2,2,2-trifluoroacetyl) -[3-(2,2,2-trifluoroacetyl-amino)propyl]amino]piperidine (0.600 g) and acetic acid (0.20 mL) in methanol (8 mL). After 5 minutes, sodium cyanoborohydride (0.220 g) in methanol (4 mL) was added in a single portion. After being stirred for 3 hours, the reaction mixture was diluted with aqueous sodium bicarbonate, stirred for 30 minutes, and extracted with dichloromethane. The organic extracts were dried, evaporated, and purified by chromatography, with dichloromethane:methanol (gradient 98:2, 90:10) as eluent. The resulting material was dissolved in dichloromethane, precipitated as the hydrochloride salt with ethereal hydrogen chloride, evaporated, and placed under high vacuum overnight to give the title compound as a white solid. MS: m/z=683(M+1).

5e. (S)-N-[4-[4-(3-Aminopropylamino)piperidino]-2-(3,4-dichlorophenyl)butyl]-N-methylbenzamide.

A solution of the (S)-N-[2-(3,4-dichlorophenyl)-4-[4-[(2,2,2-trifluoroacetyl)[3-(2,2,2-trifluoroacetylamino)propyl]amino]-piperidino]butyl]-N-methylbenzamide (2.5 g) in 20% aqueous potassium hydroxide (8.5 mL) and methanol (11 mL) was stirred for 1 hour. The reaction mixture was acidified to pH 2 with 1 N hydrochloric acid and washed 3 times with dichloromethane. The aqueous phase was then basified to pH 10 with 10 N sodium hydroxide and extracted with dichloromethane. The extracts were dried and evaporated to give the title compound as a viscous oil. MS: m/z=491(M+1).

### Example 6

The citrate salt of the compound of Example 3 was prepared as follows.

(S)-N-{2-(3,4-Dichloro-phenyl)-4-[4-(2-oxo-tetrahydro-pyrimidin-1-yl)-piperidin-1-yl]-butyl}-N-ethyl-benzamide free base (0.970 g) and citric acid (0.352 g) were dissolved in methanol and evaporated to give the title compound as a white solid. MS: m/z=531(M+1); Analysis for C₂₈H₃₆Cl₂N₄O₂.1.10 C₆H₈O₇.0.30 H₂O: Calculated: C, 55.53; H, 6.11; N, 7.48; Found: C, 55.51; H, 6.19; N, 7.47.

### Example 7

The maleate salt of the compound of Example 3 was prepared as follows.

A solution of (S)-N-{2-(3,4-dichloro-phenyl)-4-[4-(2-oxo-tetrahydro-pyrimidin-1-yl)-piperidin-1-yl]-butyl}-N-ethyl-benzamide (106.0 g) free base in isopropyl alcohol (750 mL) as added to a solution of maleic acid (23.2 g) in isopropyl alcohol (750 mL). The mixture was heated to just before reflux and then stirred at ambient temperature. Within one hour solid was forming readily. Stirred at ambient temperature overnight. The slurry was cooled in an ice bath and filtered cold, washing with chilled isopropyl alcohol. The solid was crushed and dried overnight in vacuo (250 mm at 65° C.) to yield the title compound (approximately 102 g). MS: m/z=532(M+1); Analysis for C28H36Cl2N4O2,1.0 C4H4O4: Calculated: C, 59.35; H, 6.23; N, 8.65; Found: C, 59.63-59.60; H, 6.38-6.43, N, 8.59-8.54.

The action of a compound of the Invention as a therapeutic agent for the treatment of OAB or UI through its action to bind to NK2 receptors has been shown using suitable in vitro and in vivo tests.

In Vitro Binding Assay

Preparation of Membranes from MEL Cells Transfected with Cloned Human NK1 or NK2 Receptors:

The cloning of the human lung NK1 and NK2 receptors was achieved as described by Hopkins, et al., Biochem. Biophys. Res. Commun. 180: 1110-1117 (1991), and Graham, et al., Biochem. Biophys. Res. Commun. 177: 8-16 (199.1). Heterologous expression and scale-up growth of MEL cells transfected with human tachykinin receptors was performed as described for NK2 receptors by Aharony, et al., Mol. Pharmacol. 45: 9-19, 1994.

Membranes from recombinant MEL cells expressing NK1 or NK2 receptors were prepared as described by Hopkins, et al., (1991). Briefly, cells were homogenized at 4° C. (Brinkman PT-20 Polytron, setting 3, with one 15 sec burst on ice), in a buffer consisting of 50 mM Tris-HCl (pH7.4), 5 mM KCl, 120 mM NaCl, 10 mM EDTA and containing several protease inhibitors (1 mM phenylmethylsulfonylfluoride; 0.1 mg/ml soybean trypsin inhibitor, and 1 mM iodoacetamide). The homogenate was centrifuged at 1200×g for 45 min at 4° C. to remove cell debris. The supernatant was centrifuged at 48,000×g for 45 min at 4° C. The pellet was resuspended with a glass-Teflon motorized homogenizer in 30 volumes of ice-cold 50 mM Tris-HCl (pH 7.4) buffer.

Receptor Binding Assays:

Ligand binding assays with [³H]NKA in MEL cells expressing cloned NK2 receptors or (³H]SP in MEL cells expressing cloned NK1 receptors, were conducted generally as described by Aharony, et al., Mol. Pharmacol. 45: 9-19, 1994, Aharony, et al., Neuropeptides 23: 121-130 (1992) and Aharony, et al., J. Pharmacol. Exp. Ther. 259: 146-155 (1991). In brief, incubations were carried out in assay buffer containing membranes, test compounds, and [³H] ligand (1.0-1.5 nM). In competition experiments, mixtures (0.315 mL) containing various concentrations of competing agents (agonists, antagonists, or vehicle), were incubated at 25° C. for 30 min., with or without 1 μM unlabeled homogenous ligand (NKA or SP), to define non-specific binding. Reactions were initiated by adding membranes (0.1-0.15 mg protein/final concentration) and were conducted in duplicate. Saturation and kinetic experiments were conducted in triplicate. Separation of receptor-bound and free ligand was accomplished by dilution with 1 mL of wash buffer (20 mM Tris-HCl, pH 7.5) followed immediately by vacuum filtration with a total volume of 10 mL of wash buffer (utilizing a Brandel Cell Harvester MB-48R with Whatman GF/B filters presoaked in 0.1% polyethylenimine).

The ability of compounds disclosed herein to inhibit the binding of [³H] ligand is shown by the results disclosed in Table 1.

In Vivo Assay:

BANK-Induced Bladder Contraction in Anesthetized Guinea Pigs:

Female guinea pigs (300-450 g) were anesthetized by intramuscular administration of ketamine/xylazine mixture (3/10 mg/kg, respectively). The jugular vein was catheterized and the distal end of the catheter connected to a syringe for administration of compound where appropriate. Subsequently, the bladder was exposed through a midline abdominal incision, the ureters tied with 4-0 silk suture approximately 2 cm above the bladder, and cut above the ligature to allow drainage from the kidneys. Cannula were passed through the proximal urethra and bladder sphincter into the bladder lumen. The bladder was manually emptied, infused with 0.3 mL saline, and the catheter attached to a Gould p23 ID pressure transducer for recording changes in bladder pressure. An equilibration period of approximately 15 min was allowed for stabilization of the animals following surgical preparation. Thiorphan (10 mg/kg iv) was administered 15 minutes before agonist exposure to inhibit neutral endopeptidase 3.4.24.11.

To establish the oral effect of test compounds, animals were administered the test compounds (52 nmol/kg, 5% PEG 400-saline vehicle) by gavage 1 hr before administration of BANK. Changes in bladder contraction occurring in the presence and absence of test compound were recorded as an increase in intravesical bladder pressure on a Grass 7D Polygraph and expressed as the precentage change in response. Duration of action studies were performed following oral administration of test compounds (52 nmol/kg, 5% PEG 400-saline vehicle) at different times prior to administration of BANK. Responses were calculated as the percentage difference between the response to BANK in the presence of test compound compared with sham-treated controls. For all studies, each animal was administered a single dose of test compound. Experimental results were expressed as the mean plus or minus the Standard Error of the Mean (±S.E.M) percentage change from basal level.

The ability of compounds disclosed herein to inhibit bladder contractions induced with BANK is shown by the results disclosed in Table 1.

TABLE 1

| | Inhibition of BANK- | | |
|---|---|---|---|
| | mediated GP bladder | | |
| | contraction | | |
| | (% Inhibition mediated | hNK2 | hNK1 |
| Compound of | by 52 nmol/kg | (Ki expressed | (Ki expressed |
| Example: | administered orally) | as -Log Molar) | as -Log Molar) |
| 1 | 27 ± 13 | 9.26 | 90% inhibition |
| | | | at 10 μM |
| 2 | 26 ± 16 | 9.61 | 6.95 |
| 3 | 64 ± 6 | 8.85 | 7.18 |
| 4 | 55 ± 11 | 8.76 | 7.21 |
| 5 | −9 ± 39 | 8.86 | 6.60 |

Compounds of the invention are specific for NK2 receptors. Compounds disclosed herein generally exhibit 100 fold or better selectivity for human NK2 receptors as compared to human NK1 receptors, as illustrated by the results shown in Table 1.

Surprisingly, it has been found that compounds with similar binding affinities for human NK2 receptors have different effects when tested for their ability to inhibit bladder contraction induced by BANK. For example, the compounds of Examples 3 and 5 respectively have Ki's of 8.85 and 8.86-Log Molar when tested for their ability to inhibit the binding of tritiated NKA to cloned and expressed hNK2 receptors. However, the compound of Example 3 is found to provide a 64% inhibition of BANK induced bladder contraction whereas, unexpectedly, the compound of Example 5 is found to increase the bladder contraction induced by BANK.

The Compounds of the invention have not been found to show any indication of any untoward side-effects in laboratory test animals at several multiples of the minimum effective dose.

1. A method for treating or preventing overactive bladder or urinary incontinence in a subject comprising the administration to said subject of a therapeutically-effective amount of a compound in accord with structural diagram I: 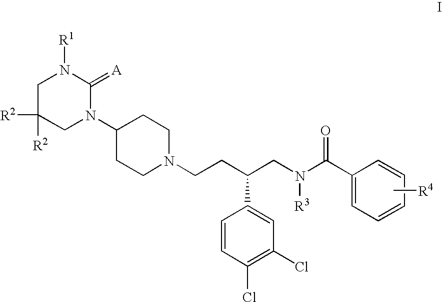
wherein,
A is O or S;
R¹ is selected from H or C₁₋₄alkyl;
R² moieties are independently selected from H or C₁₋₄alkyl;
R³ is selected from C₁₋₄alkyl;
R⁴ is selected from halogen, C₁₋₄alkyl, C₁₋₄alkoxy or cyano,
or a pharmaceutically-acceptable salt thereof with the proviso that R³ is not methyl when R¹,
R² and R are all H.

2. The method according to claim 1, comprising the administration of a compound wherein, A is O, R¹ and R² are all H, R³ is C₁₋₄alkyl, and R⁴ is selected from H or halo are useful for the treatment and prevention OAB and UI with the proviso that R³ is not methyl when R⁴ is H.

3. The method according to claim 1, comprising the administration of a compound wherein, A is O, R¹, R² and R⁴ are all H, and R³ is C₂₋₄alkyl.

4. The method according to claim 1, comprising the administration of a compound selected from:
(S)-N-[2-(3,4-dichlorophenyl)-4-[4-(2-oxo-5,5-dimethyl-perhydropyrimidin-1-yl)piperidino]butyl]-N-methylbenzamide;
(S)-N-[2-(3,4-dichlorophenyl)-4-[4-(3-ethyl-2-oxoperhydro-pyrimidin-1-yl)-piperidino]butyl]-N-methylbenzamide;
(S)-N-[2-(3,4-dichlorophenyl)-4-[4-(2-oxoperhydro-pyrimidin-1-yl)piperidino]-butyl]-N-ethylbenzamide, and
(S)-N-[2-(3,4-dichlorophenyl)-4-[4-(2-oxoperhydro-pyrimidin-1-yl)piperidino]butyl]-N-fluoro-N-methylbenzamide.

5. The method according to claim 1, wherein said subject is a human.

6. The method according to claim 1, wherein said pharmaceutically-acceptable salt is selected from the group consisting of a chloride, a sulphate, a tosylate, a mesylate, a napsylate, a besylate, a phosphate, a salicylate, a tartrate, a lactate, a citrate, a benzoate, a succinate, and acetate and a maleate.

7. The method according to claim 1, additionally comprising co-administering one or more other medically-compatible therapeutic agents.

8. The method according to claim 7, wherein said other therapeutic agents are selected from, an estrogenic agent, a progestational substance, an alpha-adrenergic agonist, a beta-adrenergic receptor blocking agent, a cholinergic-receptor blocking compound or a cholinergic-receptor-stimulating drug.

9. The method according to claim 1, wherein said compound or pharmaceutically-acceptable salt thereof is administered in a physiologically-acceptable manner, selected from topical application, ingestion, inhalation, insufflation or injection.

10. The method according to claim 9, wherein said compound or pharmaceutically-acceptable salt thereof is administered topically.

11. The method according to claim 10, comprising topically administering about 0.1 mg/kg to about 5 mg/kg of said compound or pharmaceutically-acceptable salt thereof.

12. The method according to claim 10, comprising topically administering a tablet or capsule containing about 0.1 mg up to about 250 mg of said compound or pharmaceutically-acceptable salt thereof.

13. The method according to claim 9, comprising administering by inhalation a daily dose range of 5 to 100 mg of said compound or pharmaceutically-acceptable salt thereof, in a single dose or divided into two, three or four daily doses.

14. The method according to claim 9, comprising administering about 0.01 to about 25 mg/kg of said compound or pharmaceutically-acceptable salt thereof.

15. The method according to claim 1, wherein said therapeutically-effective amount is from about 0.1 mg to about 250 mg of said compound or pharmaceutically-acceptable salt thereof, administered one to four times daily.

16. The method according to claim 15, wherein said therapeutically-effective amount is from about 5 mg to about 100 mg of said compound or pharmaceutically-acceptable salt thereof.

17. The method according to claim 1, wherein said compound or pharmaceutically-acceptable salt thereof is administered as a capsule, a tablet, an aqueous solution, an aqueous suspension, a non-aqueous suspension, a suppository, an aerosol or a powder.

18. A pharmaceutical composition for treating or preventing overactive bladder or urinary incontinence comprising (S)-N-[2-(3,4-dichlorophenyl)-4-[4-(2-oxoperhydropyrimidin-1-yl)piperidino]butyl]-N-methylbenzamide or pharmaceutically-acceptable salt thereof and at least one pharmaceutically-acceptable excipient or diluent.

19. The use of (S)-N-[2-(3,4-dichlorophenyl)-4-[4-(2-oxoperhydropyrimidin-1-yl)piperidino]butyl]-N-methylbenzamide or a pharmaceutically-acceptable salt thereof in the preparation of a medicament for treating or preventing overactive bladder or urinary incontinence.
